(19) United States
(12) Patent Application Publication
(10) Pub. No.: US 2025/0082295 A1
(43) Pub. Date: Mar. 13, 2025

(54) MEDICAL INFORMATION PROCESSING APPARATUS, RECORDING MEDIUM, AND MEDICAL INFORMATION PROCESSING METHOD

(71) Applicant: Konica Minolta, Inc., Tokyo (JP)

(72) Inventors: Kazuhiro Ono, Osaka (JP); Yuki Tomiyama, Tokyo (JP); Masaki Sunagawa, Osaka (JP)

(73) Assignee: Konica Minolta, Inc., Tokyo (JP)

(21) Appl. No.: 18/826,679

(22) Filed: Sep. 6, 2024

(30) Foreign Application Priority Data
Sep. 7, 2023 (JP) 2023-145154

(51) Int. Cl. A61B 6/00

(52) U.S. Cl. CPC A61B 6/542; A61B 6/487

(57) ABSTRACT

A medical information processing apparatus includes a hardware processor. The hardware processor, acquires at least one of performance information of dose information irradiated in one examination and a number of still images imaged in one examination, calculates a number of times of imaging based on at least one of the acquired performance information and the acquired number of still images, and outputs the calculated number of times of imaging.

### BACKGROUND OF THE INVENTION

### Technical Field

The present invention relates to a medical information processing apparatus, a recording medium, and a medical information processing method.

### Description of Related Art

In the medical field, examination apparatuses (modalities), such as computed tomography (CT) apparatuses, which irradiate an object with radiation to generate a radiation image as a medical image are used. In an examination using such an examination apparatus, it is necessary to grasp a dose of radiation used in the examination in order to appropriately manage an exposure dose of radiation.

For example, Japanese Patent No. 3597677 describes that an X-ray technician fills in imaging conditions (a tube voltage value, a tube current value, an irradiation time (amount of time), an irradiation distance, and the like) line by line in an imaging condition entry field of an imaging request sheet (an irradiation record) for each imaging.

However, in the conventional technology including the invention described in Japanese Patent No. 3597677, since the number of times of imaging is handwritten in an irradiation record, there is a possibility that an input error or the like of the number of times of imaging occurs. In this case, there is a problem that the actual number of times of imaging cannot be correctly grasped.

### SUMMARY OF THE INVENTION

An object of the present invention is to provide a medical information processing apparatus, a recording medium, and a medical information processing method that can grasp the number of times of imaging in radiation imaging more accurately than before.

In order to achieve the above object, an invention of a medical information processing apparatus includes a hardware processor, wherein the hardware processor, acquires at least one of performance information of dose information irradiated in one examination and a number of still images imaged in one examination, calculates a number of times of imaging based on at least one of the acquired performance information and the acquired number of still images, and outputs the calculated number of times of imaging.

According to another aspect, a non-transitory computer-readable recording medium storing a program that causes a computer in a medical information processing apparatus to perform the following: acquiring at least one of performance information of dose information irradiated in one examination and a number of still images imaged in one examination; calculating a number of times of imaging based on at least one of the acquired performance information and the acquired number of still images; and outputting the calculated number of times of imaging.

According to another aspect, a medical information processing method includes: acquiring at least one of performance information of dose information irradiated in one examination and a number of still images imaged in one examination; calculating a number of times of imaging based on at least one of the acquired performance information and the acquired number of still images; and outputting the calculated number of times of imaging.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating an example of a system configuration of a medical information processing system;

FIG. 2 is a block diagram illustrating the functional configuration of a dose management apparatus;

FIG. 3 is a diagram illustrating a data configuration in a database;

FIG. 4 is a block diagram illustrating a functional configuration of a client terminal;

FIG. 5 is a flowchart illustrating a control procedure of dose management processing;

FIG. 6A is a diagram illustrating an example of performance information in information related to a radiation dose;

FIG. 6B is a diagram illustrating an example of performance information in information related to the radiation dose;

FIG. 7A is a diagram illustrating an example of an irradiation record of general imaging;

FIG. 7B is a diagram showing an example of an irradiation record of a fluoroscopic contrast examination;

FIG. 8 is a diagram illustrating an example of a screen for accepting an input of imaging content; and

FIG. 9 is a flowchart illustrating a control procedure of dose management processing according to a modification example.

### DETAILED DESCRIPTION

Hereinafter, one or more embodiments of the present invention will be described with reference to the drawings. However, the scope of the invention is not limited to the disclosed embodiments.

Hereinafter, an embodiment of a medical information processing system according to the present invention will be described. The present invention is not limited to the examples illustrated in the drawings.

### 1. Configuration of Medical Information Processing System

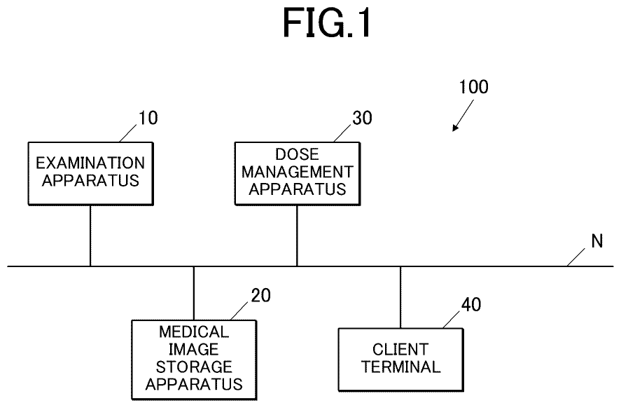

FIG. 1 illustrates an example of a system configuration of a medical information processing system 100.

As illustrated in FIG. 1, the medical information processing system 100 includes an examination apparatus 10, a medical image storage apparatus 20, a dose management apparatus 30 serving as a medical information processing apparatus, a client terminal 40, and the like.

The apparatuses included in the medical information processing system 100 are connected so as to be able to transmit and receive data via a communication network N such as a local area network (LAN) and a wide area network (WAN).

Each apparatus included in the medical information processing system 100 conforms to a HL7 (Health Level Seven) and DICOM standard. Communication between the apparatuses included in the medical information processing system 100 is performed in accordance with HL7 and DICOM.

The medical information processing system 100 may include a plurality of examination apparatuses 10 and a plurality of client terminals 40.

### 1-1. Examination Apparatus

The examination apparatus 10 is a modality such as a CT apparatus, an X-ray imaging apparatus (DR (general imaging), CR/DX (general imaging), RF (fluoroscopy imaging), or XA (angiography)), or the like.

The examination apparatus 10 operates in response to an operator's operation on a console (not illustrated).

The examination apparatus 10 irradiates a patient (a subject, a test object, or the like) with radiation, and generates image data of a radiation image (e.g., a CT image) as a medical image, based on a detection result of the radiation.

The examination apparatus 10 attaches supplementary information to the radiation image in accordance with the DICOM standard. The supplementary information includes patient information, examination information, series information, image information, and the like.

The patient information is information on a patient. The patient information includes patient ID, patient name, date of birth, sex, height, weight, age, BMI, and the like. The patient ID is identification information for specifying a patient.

The examination information is information on an examination. The examination information includes an examination ID, an examination date, an examination time (time of day), an examination description, an examination instance UID, and the like. The examination instance UID is identification information for specifying an examination, and its uniqueness is guaranteed by the DICOM standard.

The series information is information on a series. The series information includes series instance UID, series number, series date, series time (time of day), modality (CT, DR, CR/DX, RF, XA, etc), series description, and the like. The series instance UID is identification information for specifying a series, and uniqueness is guaranteed by the DICOM standard.

The image information is information on an image. The image information includes SOP instance UID, image date, image time (time of day), image number, etc. The SOP instance UID is identification information for specifying a radiation image, and uniqueness is guaranteed by the DICOM standard. The image number is a number indicating an imaging order of a tomographic image (CT image) generated by one scan.

The examination apparatus 10 generates a radiation dose structured report (RDSR) including information related to a radiation dose related to the examination performed in the examination apparatus 10. The examination apparatus 10 transmits the image data of the generated radiation image and the RDSR to the medical image storage apparatus 20 and the dose management apparatus 30.

The RDSR is information compliant with the DICOM standard, and is one of data formats of data including information related to a radiation dose of radiation.

The information related to the radiation dose is information related to the radiation dose (amount of energy) with which the object has been irradiated in a radiation examination.

The information related to the radiation dose includes an index representing the radiation dose such as an air kerma [Gy].

The information related to the radiation dose may include information of an amount correlated with the radiation dose. The information on the amount correlated with the radiation dose includes imaging conditions such as a voltage value (tube voltage value) applied for radiation irradiation, a current value (tube current value), a radiation irradiation time (amount of time), an irradiation continuous duration time, an irradiation distance (source-detector distance), a total air kerma, a total number of irradiations, an area dose, a total area dose, and a dose length product (DLP) which is a product of a CT scan length of CT examination and an exposure dose.

The information related to the radiation dose includes patient information of a patient who is the object and examination information of an examination received by the object.

The information related to the dose of radiation includes irradiation event type information indicating the type of an irradiation event, which is a batch of irradiation operation of the object with radiation during the examination. The type of the irradiation event is, for example, still image imaging or fluoroscopy.

The RDSR manages information related to these radiation doses in units of examinations.

### 1-2. Medical Image Storage Apparatus

The medical image storage apparatus 20 stores and manages the image data of the radiation image generated by the examination apparatus 10 and the information related to the radiation dose included in the RDSR for each patient and each examination. The medical image storage apparatus 20 is, for example, a picture archiving and communication system (PACS) or the like.

### 1-3. Dose Management Apparatus

The dose management apparatus 30 is a computer apparatus that manages information related to the radiation dose of the radiation image.

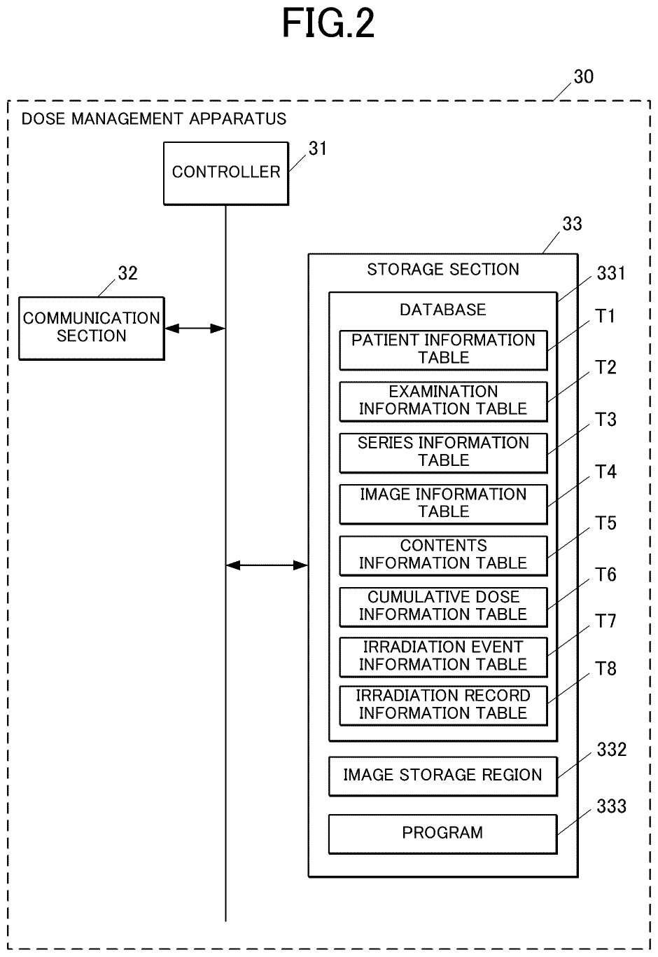

FIG. 2 is a block diagram illustrating a functional configuration of the dose management apparatus 30.

As illustrated in FIG. 2, the dose management apparatus 30 includes a controller 31 (a computer, a hardware processor), a communication section 32, a storage section 33, and the like, and each unit is connected by a bus.

The controller 31 includes a central processing unit (CPU), a read only memory (ROM), a random access memory (RAM), and the like. The controller 31 comprehensively controls processing operation of each unit of the dose management apparatus 30. Specifically, the CPU reads a program 333 stored in the storage section 33, develops the program 333 in the RAM, and performs various kinds of processing according to the program 333.

The communication section 32 includes a network interface and the like, and transmits and receives data to and from an external device connected via the communication network N. For example, the communication section 32 receives the image data of the radiation image obtained by imaging a patient and the RDSR from the examination apparatus 10. For example, the communication section 32 may receive the image data of the radiation image and the RDSR from the medical image storage apparatus 20.

The storage section 33 includes a hard disk drive (HDD), a nonvolatile semiconductor memory, and the like, and stores various types of data.

For example, the storage section 33 includes a database 331 and an image storage region 332.

The storage section 33 stores the program 333. The program 333 may be stored in the ROM of the controller 31.

The database 331 stores, in a searchable manner, supplementary information on the radiation images stored in the image storage region 332, information on the dose of radiation associated with each examination, and the like.

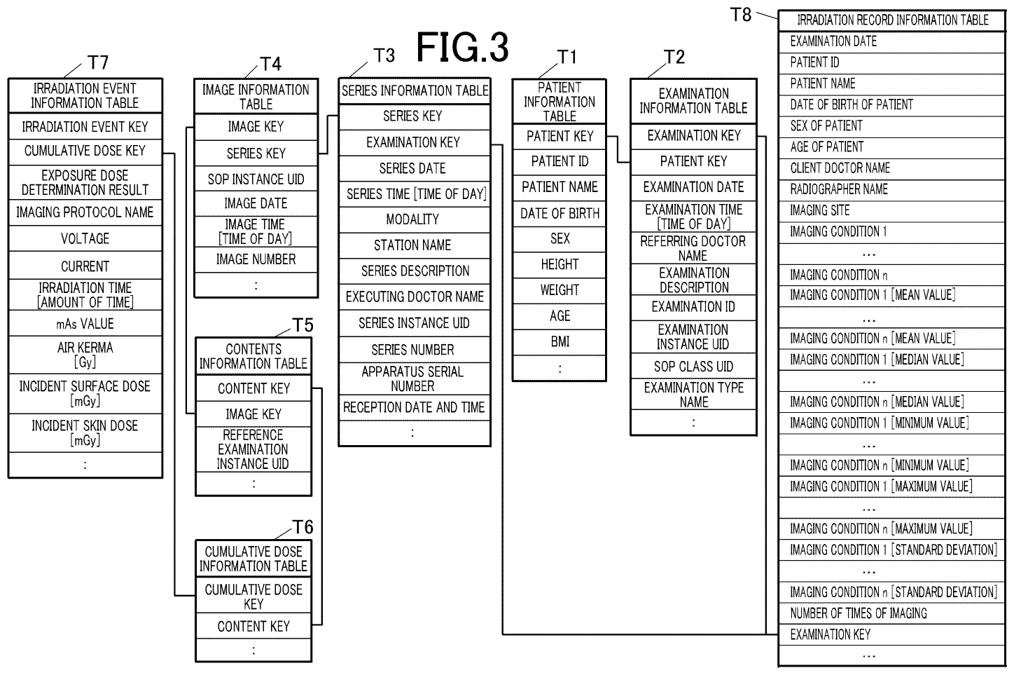

FIG. 3 illustrates a data configuration in the database 331. The database 331 includes a patient information table T1, an examination information table T2, a series information table T3, an image information table T4, a contents information table T5, a cumulative dose information table T6, an irradiation event information table T7, and an irradiation record information table T8.

The patient information table T1 stores information on patients. The controller 31 acquires the information in the patient information table T1 mainly from the RDSR, but may acquire the information from the supplementary information added to the radiation image to supplement the information as necessary.

In the patient information table T1, a patient key is used as a main key, and a patient ID, patient name, date of birth, sex, height, weight, age, BMI, and the like are associated.

The examination information table T2 stores information on examination. The controller 31 acquires the information in the examination information table T2 mainly from the RDSR, but may acquire the information from the supplementary information added to the radiation image to supplement the information as necessary.

In the examination information table T2, an examination key is used as the main key, and the patient key corresponding to a patient to be examined, an examination date, an examination time, a referring doctor name, an examination description, an examination ID, an examination instance UID, an SOP class UID, an examination type name, and the like are associated.

The series information table T3 stores information related to a series of supplementary information attached to the radiation image.

In the series information table T3, a series key is used as the main key and an examination key corresponding to an examination to which a series belongs, a series date, a series time (time of day), a modality, a station name, a series description, an executing doctor name, a series instance UID, a series number, an apparatus serial number, a reception date and time, and the like are associated. The reception date and time is a date and time when the image data of the radiation images belonging to the series is received.

The image information table T4 stores information on the image of the supplementary information attached to the radiation image.

In the image information table T4, an image key is used as the main key, and a series key corresponding to the series to which the image belongs, the SOP instance UID, the image date, the image time (time of day), the image number, and the like are associated.

The contents information table T5 stores information on the examination apparatus 10.

In the contents information table T5 a content key is used as the main key, and the image key corresponding to the image generated by the examination apparatus 10, a reference examination instance UID, and the like are associated.

The reference examination instance UID is identification information for specifying an examination in the following cases, and uniqueness is guaranteed by the DICOM standard. Specifically, the aforementioned case is a case where the examination instance UID included in the RDSR and the examination instance UID associated with the image of the radiation image generated in the examination apparatus 10 are different from each other.

The cumulative dose information table T6 stores information about a cumulative dose which is the total of air kerma in all imaging sites in units of irradiation events.

In the cumulative dose information table T6, a cumulative dose key is used as the main key, and the content key corresponding to the examination apparatus 10 that has performed examination are associated

The irradiation event information table T7 stores information (irradiation event information) of an irradiation unit generated during examination.

In the irradiation event information table T7, an irradiation event key is used as the main key, and the cumulative dose key, an exposure dose determination result, an imaging protocol name, a voltage, a current, the irradiation time (amount of time), a mAs value (current-time (amount of time) product), the air kerma [Gy], an incident surface dose [mGy], an incident skin dose [mGy], and the like corresponding to an irradiation event are associated.

The exposure dose determination result is a determination result as to whether or not there is a problem with the exposure dose of the patient. In a case where the exposure dose of the patient is larger than diagnostic reference levels (DRLs) which are reference values of general dose management, determination of NG is stored as the exposure dose determination result. When the exposure dose of the patient is equal to or lower than the diagnostic reference level, the determination of OK is stored as the exposure dose determination result. The determination of the exposure dose may be performed by the radiographer or the controller 31 of the dose management apparatus 30.

The air kerma is an air absorbed dose at a skin surface position at which a radiation dose required when a patient is assumed to be present at an irradiation position of the examination apparatus 10 is output from the examination apparatus 10. The air kerma does not include backscatter from the patient.

An entrance surface dose is the air absorbed dose including backscattering at the skin surface position of the patient.

The entrance skin dose is the absorbed dose of skin including backscattered rays from the patient.

The irradiation record information table T8 stores information on irradiation records for recording imaging conditions, imaging situations, and the like. The information on the irradiation record includes performance information in the information related to the dose of radiation emitted to the object.

The performance information in the information related to the radiation dose is, for example, information such as an examination date and time (examination date) when the patient is irradiated with radiation and imaged, a patient name, an age of the patient, a client doctor name, an imaging condition, and an air kerma. The air kerma is an actual dose of radiation in imaging.

The performance information in the information related to the radiation dose is information at the time of the end of the examination.

In the irradiation record information table T8, an examination key is used as the main key, and date of examination, patient ID, patient name, date of birth of the patient, sex of the patient, age of the patient, client doctor name, radiographer name, imaging site, imaging condition, mean value of imaging condition, median value of imaging condition, minimum value of imaging condition, maximum value of imaging condition, standard deviation of imaging condition, number of times of imaging (number of times of exposure) actually performed in the examination, and the like in the performance information are associated.

The imaging condition includes a tube voltage value, a tube current value, the irradiation time (amount of time), the irradiation distance, the mAs value, a total fluoroscopy time (amount of time), the area dose, the air kerma, the incident surface dose, and the incident skin dose.

The incident surface dose [mGy] and the incident skin dose [mGy] are stored in the irradiation record information table T8 when the irradiation event type of the imaging by the examination apparatus 10 is the fluoroscopy or the angiography. That is, in a case where the irradiation event type of the imaging by the examination apparatus 10 is the CT imaging or the general imaging, the incident surface dose [mGy] and the incident skin dose [mGy] are not stored in the irradiation record information table T8.

The total fluoroscopy time (amount of time) is stored in the irradiation record information table T8 when the irradiation event type of imaging by the examination apparatus 10 is fluoroscopy.

As illustrated in FIG. 3, the examination information table T2 is associated with the patient information table T1 via the patient key.

The series information table T3 is associated with the examination information table T2 via the examination key.

The image information table T4 is associated with the series information table T3 via the series key.

The contents information table T5 is associated with the image information table T4 via the image key.

The cumulative dose information table T6 is associated with the contents information table T5 via the content key.

The irradiation event information table T7 is associated with the cumulative dose information table T6 via the cumulative dose key.

The irradiation record information table T8 is associated with the examination information table T2 and the series information table T3 via the examination key.

Therefore, according to the database 331, it is possible to acquire the correspondence relationship between each image information table T4 and the irradiation record information table T8. In other words, according to the database 331, it is possible to acquire the correspondence relationship between each radiation image and the performance information in the information related to the radiation dose.

The image storage region 332 stores image data of the radiation image and the like.

In the image storage region 332, by dividing the folders by date, by patient, by examination, and/or by series, the controller 31 can realize high-speed access when acquiring a file of the image data or the like. Therefore, even if the controller 31 does not access the database 331, the controller 31 can specify the folder in which a target radiation image is stored, the file of the radiation image, and the like by the supplementary information added to the radiation image.

The image storage region 332 stores, for each patient and/or each examination, information included in the RDSR corresponding to the examination.

### 1-4. Client Terminal

Next, the client terminal 40 will be described. The client terminal 40 is a computer device such as a personal computer (PC) used by a doctor or a radiographer. The doctor or the radiographer browses, at the client terminal 40, the radiation image related to the examination, the information related to the radiation dose, and the like.

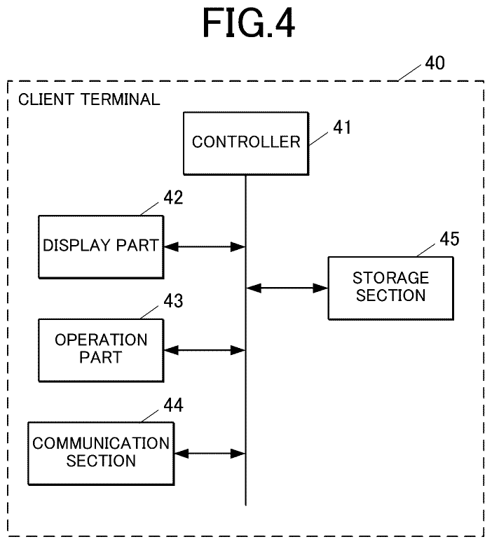

FIG. 4 is a block diagram illustrating a functional configuration of the client terminal 40.

As illustrated in FIG. 4, the client terminal 40 includes a controller 41, a display part 42, an operation part 43, a communication section 44, a storage section 45, and the like, which are connected to each other by a bus.

The controller 41 includes a CPU, a ROM, a RAM, and the like and comprehensively controls processing operation of sections of the client terminal 40. Specifically, the CPU reads various processing programs stored in the ROM, develops the read programs in the RAM, and carries out various processes in cooperation with the programs.

The display part 42 includes a monitor such as a liquid crystal display (LCD) and displays various screens according to instructions of display signals input from the controller 41.

The operation part 43 includes a keyboard having cursor keys, character input keys, number input keys, various function keys, and the like, and a pointing device such as a mouse. The operation part 43 outputs, to the controller 41, an operation signal input by a key operation on the keyboard, a mouse operation, or the like. The operation part 43 may include a touch screen overlaid on the monitor of the display part 42.

The communication section 44 includes a network interface and the like, and transmits and receives data to and from the external device connected via the communication network N.

The storage section 45 includes an HDD, a nonvolatile semiconductor memory, and the like, and stores various types of data.

### 2. Operation in Medical Information Processing System

Next, the operation of each device in the medical information processing system 100 will be described.

In the medical information processing system 100, the examination apparatus 10 executes an irradiation event to generate the radiation image. Next, the examination apparatus 10 transmits the image data of the radiation image and the RDSR related to the irradiation event to the medical image storage apparatus 20 and the dose management apparatus 30.

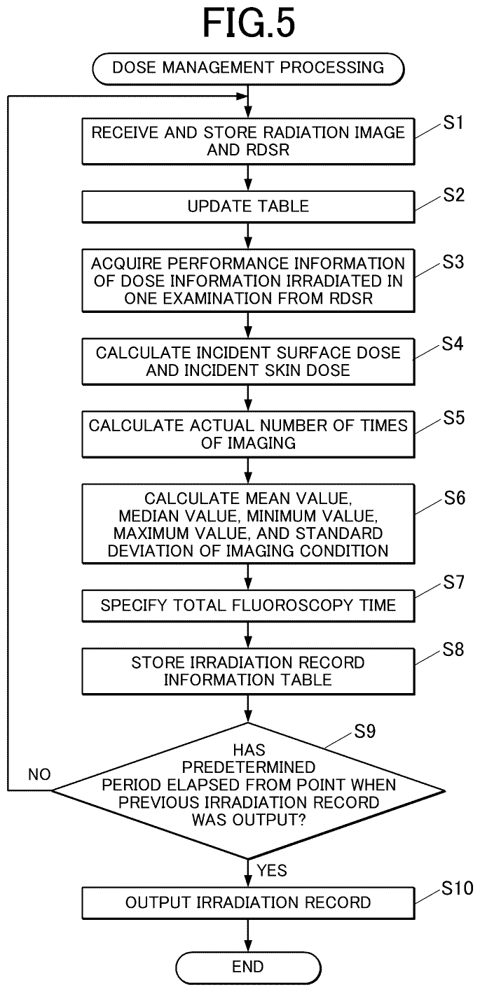

The dose management apparatus 30 executes dose management processing illustrated in FIG. 5 on the basis of the received image data of the radiation image and the RDSR.

### (Dose Management Processing)

The controller 31 of the dose management apparatus 30 receives the image data of the radiation image and the RDSR via the communication section 32. Next, the controller 31 stores the image data of the received radiation image and the information included in the RDSR in the image storage region 332 of the storage section 33 (step S1).

Next, the controller 31 updates the contents of the patient information table T1, the examination information table T2, the series information table T3, the image information table T4, the contents information table T5, the cumulative dose information table T6, and the irradiation event information table T7 on the basis of the received supplementary information on the radiation image and the information included in the RDSR (step S2).

Next, the controller 31 acquires, from the RDSR received in step S1, the performance information of the information on the amount of the radiation dose (dose information) for each examination (step S3).

That is, the controller 31 acquires the performance information of the dose information applied in one examination. The controller 31 functions as an acquisition section. Step S3 is an acquiring process.

In step S3, the controller 31 specifies the examinations having the same examination instance UID as the same examination.

The controller 31 may acquire the performance information of the dose information applied in one examination from a DICOM structured report other than the RDSR.

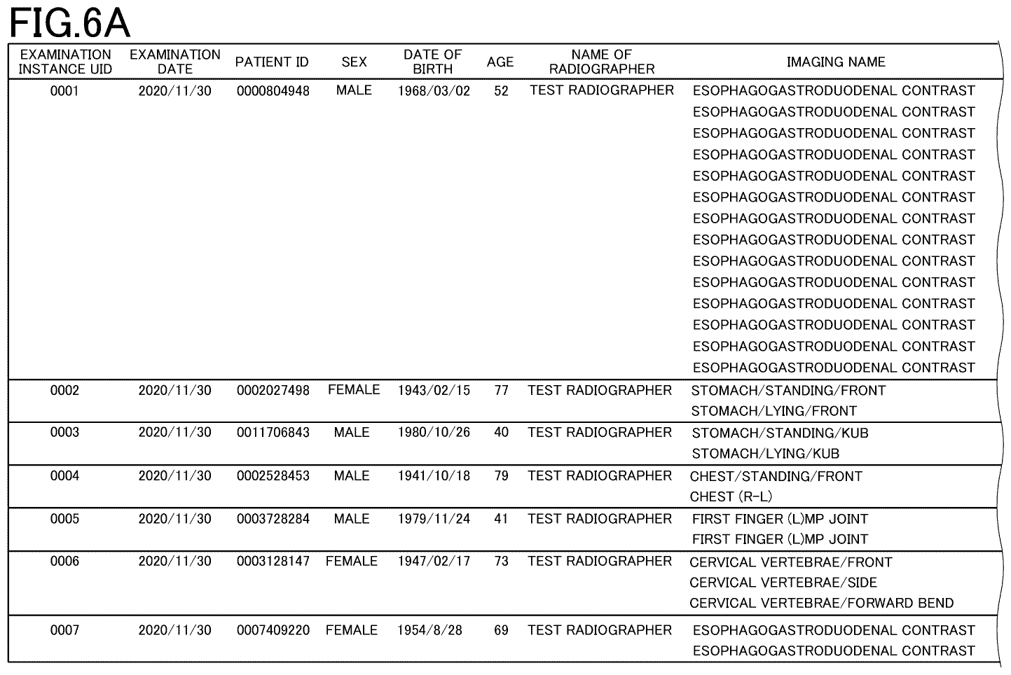

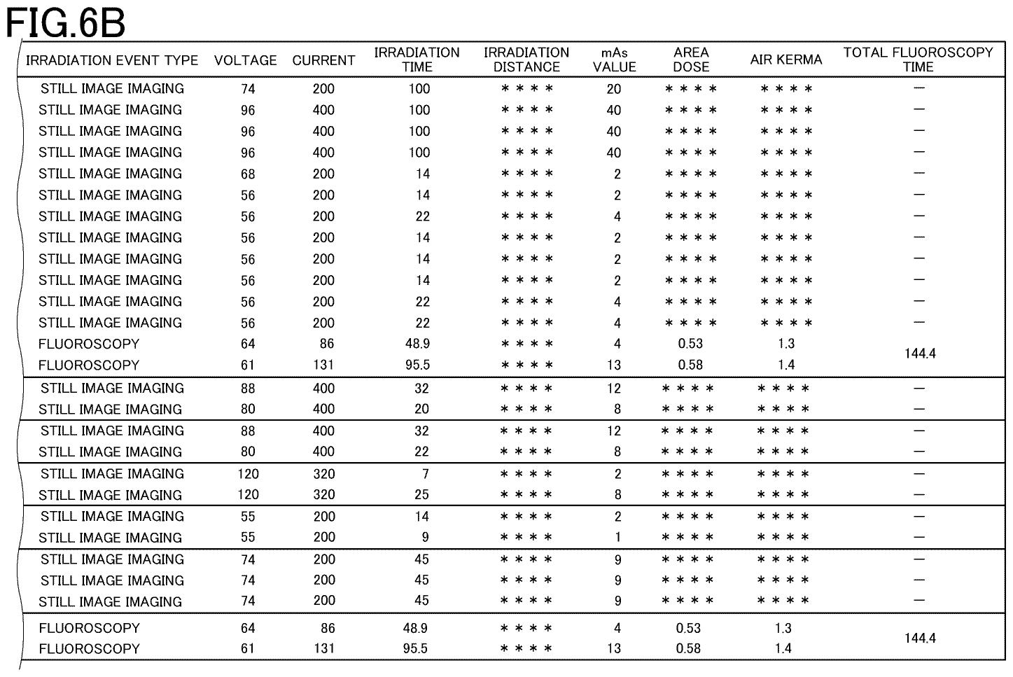

FIG. 6A and FIG. 6B illustrate an example of the performance information of the dose information for each examination. In the examples illustrated in FIG. 6A and FIG. 6B, the performance information of the dose information is described in one row for each irradiation event.

In the examples illustrated in FIG. 6A and FIG. 6B, the items of the performance information acquired in step S3 include the examination instance UID, the examination date, the patient ID, the sex of the patient, the date of birth of the patient, the age of the patient, the name of the radiographer, the imaging name (imaging site and imaging direction), the irradiation event type, the tube voltage value, the tube current value, the irradiation time (amount of time), the irradiation distance, the mAs value, area dose, and the air kerma.

Next, the controller 31 calculates the incident surface dose and the incident skin dose for an irradiation event whose irradiation event type is fluoroscopy or angiography in the performance information acquired in step S3 (step S4).

To be specific, the controller 31 calculates the incident surface dose (ESD1 [mGy]) by the following formula (1) on the basis of the air kerma (I D [Gy]) as the performance information.

$$ESD 1 = ID \times 0.85 \times 1.3 \qquad Formula ( 1 )$$

The controller 31 calculates the incident skin dose (ESD2 [mGy]) by the following formula (2) based on the air kerma (I D [Gy]) as the performance information.

$$ESD 2 = ID \times 0.85 \times 1.3 \times 1.06 \qquad Formula ( 2 )$$

When the irradiation event type is CT imaging or general imaging in the performance information acquired in step S3, the controller 31 skips step S4.

Next, the controller 31 calculates the number of times of imaging actually performed for each examination based on the performance information acquired in step S3 (step S5). The controller 31 functions as a calculation section. The step S5 is a calculation process.

To be specific, the controller 31 calculates the number of times of the irradiation event in which the irradiation event type is still-image photographing in the performance information acquired in step S3 as the actual number of times of photographing.

In the examples illustrated in FIG. 6A and FIG. 6B, the irradiation event types of the examination with the examination instance UID “0001” include still-image imaging and fluoroscopic imaging, and since the number of times of still-image imaging is 12 times and the number of times of fluoroscopic imaging is 2 times, the actual number of times of imaging is 12 times. The actual number of times of imaging in each of the examinations with the examination instance UIDs “0002”, “0003”, “0004”, and “0005” is 2 times. The actual number of times of imaging in the examination with the examination instance UID of “0006” is 3 times. In the examination with the examination instance UID of “0007”, only fluoroscopy that does not include still image imaging is performed, and thus the controller 31 does not calculate the number of times of imaging for the examination with the examination instance UID of “0007”.

When the radiographer writes the number of times of imaging in the irradiation record by hand, the radiographer may omit imaging that is not used as an examination result, such as imaging in which an imaging failure has occurred, from the number of times of imaging to be written in the irradiation record. In this case, there is a problem that it is not possible to accurately grasp the actual number of times of imaging.

However, since the RDSR also includes information associated with the radiation dose at the time of the imaging failure, in the present invention, the actual imaging count calculated in step S5 includes the imaging count of the imaging in which the imaging failure has occurred. Thus, the actual number of times of imaging in the radiation imaging can be accurately grasped.

Next, based on the performance information acquired in step S3, the controller 31 calculates at least one of mean value, median value, minimum value, maximum value, and standard deviation of the imaging condition for each examination (step S6). The imaging conditions include the tube voltage value, the tube current value, the irradiation time (amount of time), the irradiation distance, the mAs value, the area dose, the air kerma, and the incident surface dose and the incident skin dose calculated in step S4. That is, based on the performance information of the dose information irradiated in one examination, the controller 31 as the calculation section calculates at least one of the mean value, the median value, the minimum value, the maximum value, and the standard deviation of the performance information.

Next, the controller 31 specifies, as the total fluoroscopy time (amount of time) for each examination, the irradiation time (amount of time) of the irradiation event of which the irradiation event type is fluoroscopy in the performance information acquired in step S3 (step S7). The controller 31 functions as a specifying section.

In the example illustrated in FIG. 6A and FIG. 6B, the irradiation event type of the examination corresponding to the examination instance UID “0007” is fluoroscopy. Therefore, the controller 31 acquires the total fluoroscopy time (amount of time) for each examination included in the performance information of the examination.

If the irradiation event type is imaging other than fluoroscopy in the performance information acquired in step S3, the controller 31 skips step S7.

Next, the controller 31 records the performance information in the information relating to the radiation dose acquired in step S3 in the irradiation record information table T8.

In a case where step S4 is performed, the controller 31 records the incident surface dose and the incident skin dose calculated in step S4 in the irradiation record information table T8.

The controller 31 records the number of times of imaging actually performed for each examination calculated in step S5 in the irradiation record information table T8.

The controller 31 records at least one of the mean value, the median value, the minimum value, the maximum value, and the standard deviation of the imaging condition for each examination calculated in step S6 in the irradiation record information table T8.

If step S7 is performed, the controller 31 records the total fluoroscopy time (amount of time) specified in step S7 in the irradiation record information table T8 (step S8).

Next, the controller 31 determines whether a predetermined period has elapsed from the time when the previous irradiation record was output (step S9).

The predetermined period is a preset period, for example, one week or one month.

If the predetermined period has not elapsed since the previous irradiation record was output (step S9: NO), the controller 31 shifts the dose management processing to step S1.

On the other hand, the case where the predetermined period has elapsed from the time of outputting the previous irradiation record (step S9; YES) will be described. In this case, the controller 31 outputs the irradiation record including various kinds of information and the like stored in step S8 until the present time after the previous irradiation record is output (step S10), and ends the dose management processing.

That is, the controller 31 outputs the number of times of imaging actually performed for each examination. The controller 31 functions as an output section. Step S10 is an output process.

The irradiation record may include not only the performance information in the information relating to the radiation dose, the incident surface dose and the incident skin dose, the actual number of times of imaging, the mean value, the median value, the minimum value, the maximum value, and the standard deviation of the imaging conditions, and the total fluoroscopy time (amount of time), but also the radiation image relating to the performance information.

As the output of the irradiation record, the controller 31 causes, for example, the display part 42 of the client terminal 40 to display a screen for displaying the irradiation record.

The irradiation record may be in the form of a list in which information is described for each item as illustrated in FIG. 6A and FIG. 6B.

The irradiation record may be output in a format such as portable document format (PDF) and Microsoft Excel (registered trademark).

As the output of the irradiation record, the controller 31 may print the irradiation record on a paper medium. In this case, the controller 31 prints the irradiation record in the format of PDF, Microsoft Excel, or the like.

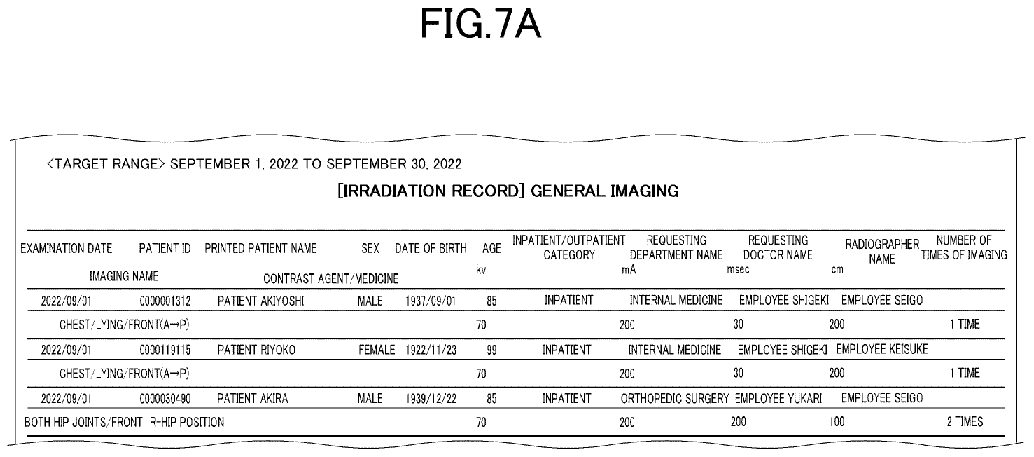

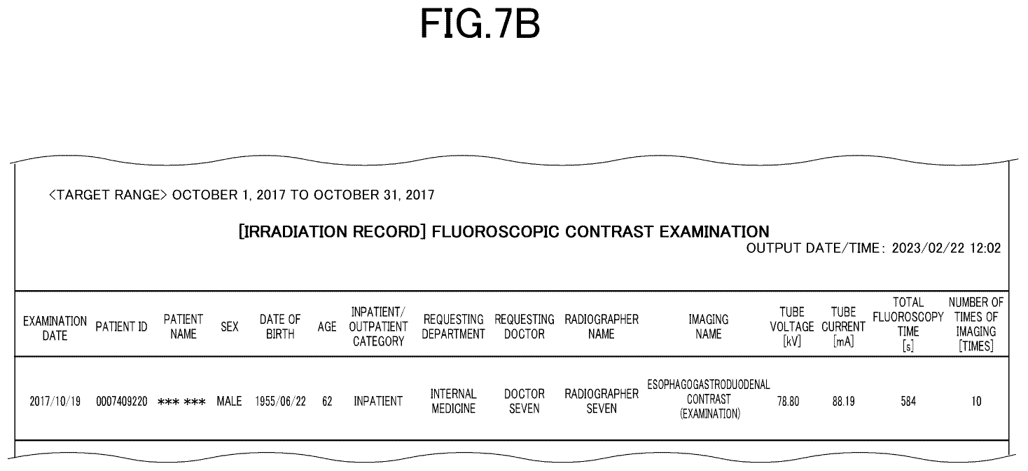

FIG. 7A shows an example of the irradiation record in general imaging. FIG. 7B shows an example of the irradiation record of fluoroscopic contrast examination.

In the example illustrated in FIG. 7A and FIG. 7B, the controller 31 displays, in the irradiation record, date of examination, patient ID, patient name, patient sex, patient date of birth, patient age, inpatient/outpatient category, requesting doctor name, radiographer name, imaging name, and the like.

In the example illustrated in FIG. 7A, the controller 31 displays, in the irradiation record, the mean value of the imaging conditions (tube voltage value, tube current value, irradiation time (amount of time), and irradiation length) for each examination, the number of times of actual imaging, and the like.

In the example illustrated in FIG. 7B, the controller 31 displays, in the irradiation record, the mean value of the imaging conditions (tube voltage value and tube current value) for each examination, the total fluoroscopy time (amount of time), the actual number of times of imaging, and the like.

As illustrated in FIG. 7A and FIG. 7B, the controller 31 displays, in the irradiation record, the mean value of the imaging conditions for each examination and the actual number of times of imaging, thus allowing the user to grasp the approximate irradiation dose per examination.

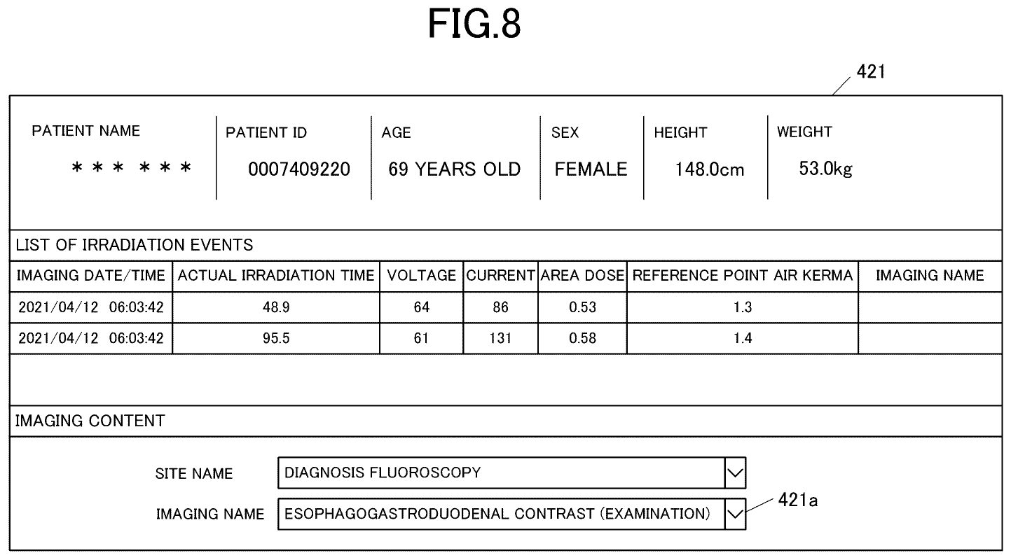

A case will be described in which the imaging content such as the imaging name is not included in the performance information acquired in step S3 of the dose management processing. In this case, the controller 31 causes the display part 42 of the client terminal 40 to display an acceptance screen 421 (see FIG. 8) for accepting the input of the imaging content.

The controller 31 accepts input of imaging content such as the imaging name by the user such as the radiographer in an input field 421a of the acceptance screen 421.

That is, the controller 31 accepts the input of the imaging content when the performance information of the dose information irradiated in one examination does not include the information relating to the imaging content. The controller 31 functions as a reception section.

The controller 31 as the output section outputs the irradiation record including the imaging content such as the imaging name whose input has been accepted as the acceptance section.

The performance information acquired in step S3 and output in step S10 may include performance information on the X-ray tube current, the total air kerma, and the total area dose. Information on the X-ray tube current, the total air kerma, and the total area dose is not included in a DICOM MPPS (Modality Performed Procedure Step). Therefore, by executing the dose management processing, it is possible to output an irradiation record including the performance information of the X-ray tube current, the total air kerma, and the total area dose, which is not included in the MPPS. Therefore, the user can grasp the performance information in the information related to a larger radiation dose.

The performance information acquired in step S3 and output in step S10 may include performance information on DLP in the CT examination. The information of the DLP is information included only in the RDSR. Therefore, by executing the dose management processing, it is possible to output an irradiation record including the performance information of the DLP. Therefore, the user can grasp the performance information in the information related to a larger radiation dose.

As described above, in the RDSR, information related to the radiation dose is managed for each examination. Therefore, in step S3, the controller 31 can collectively acquire, for each examination, the performance information in the information related to the radiation dose from the RDSR. On the other hand, in a case where the information related to the radiation dose is acquired from the attribute information of the DICOM image, it is necessary to confirm all the captured images and acquire the information related to the radiation dose of the target. Therefore, by executing the dose management processing, it is possible to grasp the information related to the radiation dose more easily than acquiring the information related to the radiation dose from the attribute information of the DICOM image.

The RDSR also includes information related to the radiation dose at the time of imaging failure. Therefore, in step S3, the controller 31 acquires the performance information in the information related to the radiation dose at the time of imaging failure from the RDSR. On the other hand, the attribute information of the DICOM image cannot be acquired at the time of imaging failure. Therefore, by executing the dose management processing, there is an advantage that the information related to the radiation dose can be grasped even at the time of imaging failure, as compared with a case where the information related to the radiation dose is acquired from the attribute information of the DICOM image.

As described above, the irradiation record is output every predetermined period, and thus it is possible to easily perform output management of the irradiation record. In a case where the irradiation record is printed on a paper medium for each predetermined period, the amount of the paper medium can be reduced as compared with a case where the irradiation record is printed for each irradiation event.

In the above dose management processing, the case where the dose management apparatus 30 receives the radiation image and the RDSR from the examination apparatus 10 has been described. However, the dose management apparatus 30 may receive the radiation image and the RDSR from the medical image storage apparatus 20. The timing at which the radiation image and the RDSR are received from the examination apparatus 10 or the medical image storage apparatus 20 does not have to be the same timing.

### 3. Modification Examples

Next, a modification example of the medical information processing system 100 described in the above embodiment will be described. The differences from the above-described embodiment will be mainly described below.

The medical information processing system 100 of the present modification example has the same configuration as the medical information processing system 100 of the above-described embodiment.

The medical image storage apparatus 20 of the present modification example receives the image data of the radiation image generated by the examination apparatus 10. Next, the medical image storage apparatus 20 counts the number of still images in the received radiation images for each examination. Next, the medical image storage apparatus 20 stores the counted number of still images for each examination. The medical image storage apparatus 20 determines that the radiation images having the same examination instance UID included in the supplementary information of the radiation images are the radiation images captured in the same examination.

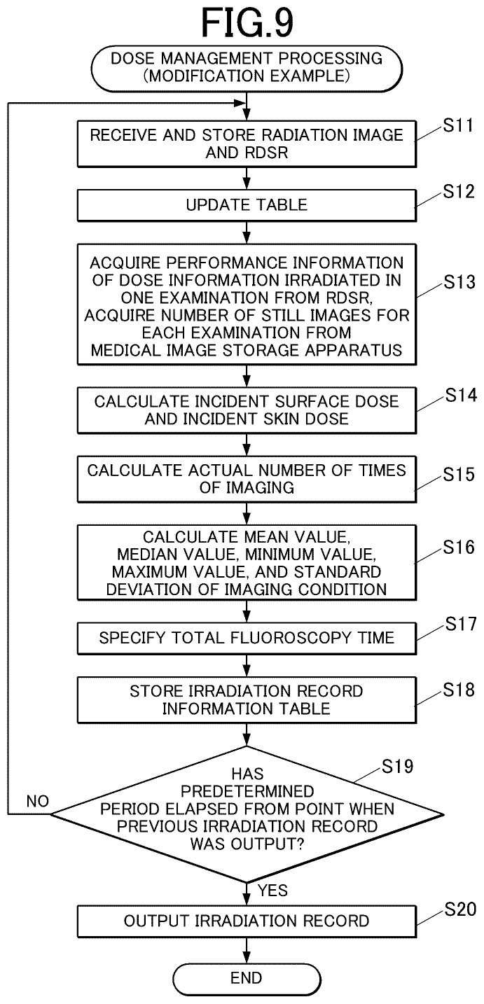

FIG. 9 shows a flowchart of the dose management processing of the present modification example.

### Dose Management Processing of Modification Example

The controller 31 of the radiation dose management apparatus 30 executes steps S11 and S12 similar to the radiation dose management processing steps S1 and S2 of the above-described embodiment.

Next, the controller 31 acquires performance information of information (dosage information) regarding the dose of radiation for each examination from the RDSR received in step S11. The controller 31 acquires the number of still images for each examination from the medical image storage apparatus 20 (step S13).

That is, the controller 31 serving as an acquisition section acquires the number of still images captured in one examination. Step S13 is an acquiring process.

Next, the controller 31 executes step S14 similar to step S4 of the radiation dose management processing of the above-described embodiment.

Next, the controller 31 calculates the number of still images acquired in step S13 for each examination as the actual number of times of imaging for each examination (step S15).

That is, the controller 31 as a calculation section calculates the actual number of times of imaging based on the acquired number of still images captured in one examination. Step S15 is a calculation process.

Next, the controller 31 executes steps S16 to S20 similar to steps S6 to S10 of the radiation dose management processing of the above-described embodiment, and ends the radiation dose management processing.

### 4. Effect

As described above, the medical information processing apparatus (the dose management apparatus 30) included in the medical information processing system 100 according to the present embodiment includes an acquisition section (the controller 31) that acquires at least one of the performance information of the dose information irradiated in one examination and the number of still images captured in one examination.

The medical information processing apparatus includes a calculation section (controller 31) that calculates the number of times of imaging on the basis of at least one of the performance information and the number of still images acquired by the acquisition section.

The medical information processing apparatus includes an output section (controller 31) that outputs the number of times of imaging calculated by the calculation section.

Thus, since the actual number of times of imaging calculated by the dose management apparatus 30 can be output, the number of times of imaging in radiation imaging can be grasped more accurately than before.

The acquisition section (the controller 31) included in the medical information processing apparatus (the dose management apparatus 30) according to the present embodiment acquires performance information of dose information irradiated in one examination.

A calculation section (controller 31) included in the medical information processing apparatus calculates the number of times of imaging based on the performance information.

Thus, since the actual number of times of imaging calculated by the dose management apparatus 30 can be output based on the performance information of the dose information, the number of times of imaging in the radiation imaging can be grasped more accurately than before.

The acquisition section (the controller 31) included in the medical information processing apparatus (the dose management apparatus 30) according to the present embodiment acquires performance information of dose information irradiated in one examination.

The calculation section (controller 31) of the medical information processing apparatus calculates, based on the performance information, at least one of the mean value, the median value, the minimum value, the maximum value, and the standard deviation of the performance information.

Thus, at least one of the mean value, the median value, the minimum value, the maximum value, and the standard deviation of the performance information such as the imaging conditions in the radiography can be grasped.

The calculation section (controller 31) included in the medical information processing apparatus (dose management apparatus 30) according to the present embodiment calculates, as the number of times of imaging, the number of times of the irradiation event in which the irradiation event type in the performance information is still image imaging.

Thus, the actual number of times of imaging can be easily calculated.

The medical information processing apparatus (the dose management apparatus 30) according to the present embodiment includes a specifying section (the controller 31) that specifies, as the total fluoroscopy time (amount of time), the irradiation time (amount of time) of the irradiation event of which the irradiation event type in the performance information is fluoroscopy.

Thus, the total fluoroscopy time (amount of time) in fluoroscopy can be easily specified.

The medical information processing apparatus (the dose management apparatus 30) according to the present embodiment includes an acceptance section (the controller 31) that accepts an input of imaging content in a case where information regarding the imaging content is not included in the performance information.

The output section (controller 31) of the medical information processing apparatus (dose management apparatus 30) outputs the imaging content accepted by the acceptance section.

Thus, since the imaging content can be output even in a case where the performance information does not include the information on the imaging content, the user can grasp the imaging content in the irradiation record.

In the medical information processing apparatus (dose management apparatus 30) according to the present embodiment, the number of times of imaging includes the number of times of imaging in which an imaging failure has occurred.

This makes it possible to accurately grasp the actual number of times of imaging including the number of times of imaging in which an imaging failure has occurred.

The description in the above embodiment is an example of the medical information processing system according to the present invention, and the present invention is not limited to this. The detailed configuration and the detailed operation of each part constituting the system can be appropriately changed without departing from the spirit of the present invention.

For example, although the controller 31 outputs the irradiation record every predetermined period in the dose management processing of the above embodiment, it is not limited thereto. The controller 31 may output the irradiation record each time the irradiation event occurs in the examination apparatus 10, that is, each time the radiation image and the RDSR are received.

In a case where the dose management apparatus 30 is provided with the operation part and the display part and the user can directly operate the dose management apparatus 30, the dose management apparatus 30 may accept the user operation. In this case, the screen for displaying the irradiation record, which is displayed on the display section 42 of the client terminal 40 in the above-described embodiment, is displayed on the display part of the dose management apparatus 30.

In the dose management process of the above-described embodiment, the controller 31 automatically outputs the irradiation record based on the performance information in the information relating to the radiation dose, but the invention is not limited thereto. Upon accepting an instruction to output the irradiation record through the user operation, the controller 31 may output the irradiation record.

The medical image storage apparatus 20 may have the function of the dose management apparatus 30. In this case, the medical image storage apparatus 20 corresponds to the “medical information processing apparatus” of the present invention.

The above embodiment has been described using the example in which the storage section 33 of the dose management apparatus 30 is provided with the database 331 and the image storage region 332, but is not limited thereto. For example, the database 331 may be provided in a database server or the like outside the dose management apparatus 30, and the controller 31 may acquire necessary data from the database server each time. Similarly, the controller 31 may store image data of the radiation image in the external storage apparatus of the dose management apparatus 30, and acquire necessary image data from the storage apparatus each time.

In the database 331, the irradiation record information table T8 may include the series key, and the irradiation record information table T8 and the image information table T4 may be associated with each other via the series key. For such management of the database 331, the RDSR may include the series instance UID to be associated with the radiation image.

Although an example in which the storage section 33 or the ROM is used as a computer-readable medium storing the program for executing each processing has been disclosed in the above description, the present invention is not limited to this example. As other computer-readable media, a nonvolatile memory such as a flash memory and a portable recording medium such as a CD-ROM are also applicable. As a medium for providing program data via a communication line, a carrier wave may be used.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-145154, filed on Sep. 7, 2023, including description, claims, drawings and abstract is incorporated herein by reference.

What is claimed is:

1. A medical information processing apparatus comprising:
a hardware processor,
wherein the hardware processor,
acquires at least one of performance information of dose information irradiated in one examination and a number of still images imaged in one examination,
calculates a number of times of imaging based on at least one of the acquired performance information and the acquired number of still images, and
outputs the calculated number of times of imaging.

2. The medical information processing apparatus according to claim 1, wherein the hardware processor, acquires performance information of the dose information irradiated in the one examination, and calculates the number of times of imaging based on the performance information.

3. The medical information processing apparatus according to claim 1, wherein the hardware processor, acquires the performance information of the dose information irradiated in the one examination, and on the basis of the performance information, calculates at least one of a mean value, a median value, a minimum value, a maximum value, and a standard deviation of the performance information.

4. The medical information processing apparatus according to claim 2, wherein the hardware processor, calculates, as the number of times of imaging, a number of times of an irradiation event in which an irradiation event type in the performance information is still image imaging.

5. The medical information processing apparatus according to claim 2, wherein the hardware processor, specifies, as a total fluoroscopy time, an irradiation time of an irradiation event whose irradiation event type in the performance information is fluoroscopy.

6. The medical information processing apparatus according to claim 1, wherein the hardware processor,
accepts input of imaging content in a case in which the performance information does not include information regarding imaging content, and
outputs the accepted imaging content.

7. The medical information processing apparatus according to claim 1, wherein the number of times of imaging includes a number of times of imaging in which an imaging failure occurred.

8. A non-transitory computer-readable recording medium storing a program that causes a computer in a medical information processing apparatus to perform the following:
acquiring at least one of performance information of dose information irradiated in one examination and a number of still images imaged in one examination;
calculating a number of times of imaging based on at least one of the acquired performance information and the acquired number of still images; and
outputting the calculated number of times of imaging.

9. A medical information processing method comprising:
acquiring at least one of performance information of dose information irradiated in one examination and a number of still images imaged in one examination;
calculating a number of times of imaging based on at least one of the acquired performance information and the acquired number of still images; and
outputting the calculated number of times of imaging.
